(19) United States
(12) Patent Application Publication
(10) Pub. No.: US 2025/0163008 A1
(43) Pub. Date: May 22, 2025

(54) A METHOD FOR TREATING ROSAH SYNDROME

(71) Applicant: Shanghai Yao Yuan Biotechnology Co., Ltd., Shanghai (CN)

(72) Inventors: Danyang Liu, Shanghai (CN); Cong Xu, Shanghai (CN); Lawrence S. Melvin, Shanghai (CN); Xiong Wei, Shanghai (CN); Tongruei Raymond Li, Shanghai (CN); Jieqing Fan, Shanghai (CN); Yanfang Pan, Shanghai (CN); Huaixin Dang, Shanghai (CN); Henri Lichenstein, Guilford, CT (US); Tian Xu, Shanghai (CN)

(21) Appl. No.: 19/036,564

(22) Filed: Jan. 24, 2025

Related U.S. Application Data
(63) Continuation of application No. 18/246,586, filed on Mar. 24, 2023.

(51) Int. Cl. C07D 277/44; A61P 13/12; C07D 233/88; C07D 263/48; C07D 417/12; C07D 487/18

(52) U.S. Cl. CPC C07D 277/44; A61P 13/12; C07D 233/88; C07D 263/48; C07D 417/12; C07D 487/18

(57) ABSTRACT

The present invention discloses a method for treating ROSAH syndrome, specifically relates to a method for inhibiting ALPK1 or ALPK1^{[T237M]}. The ALPK1 inhibitor has excellent pharmacokinetic properties, and has the potential to serve as a precision-targeted drug for the treatment of ROSAH syndrome.

### CROSS-REFERENCE TO RELATED APPLICATIONS

The present application is a Continuation-in-part Application of U.S. application Ser. No. 18/246,586, filed on Mar. 24, 2023, which is a National Stage of International Application No. PCT/CN2021/119801, filed on Sep. 23, 2021, which claims priority of International Application No. PCT/CN2020/117436, filed on Sep. 24, 2020.

### REFERENCE TO SEQUENCE LISTING

The present application contains a Sequence Listing as an XML file entitled “BSP24417751US-CIP-2-SEQ” created date of Oct. 29, 2024 and having a size of 15,454 bytes.

### TECHNICAL FIELD

The present invention relates to a method for treating ROSAH syndrome.

### BACKGROUND

ROSAH (retinal dystrophy, optic nerve edema, splenomegaly, anhidrosis, and headache) syndrome is an autosomal dominant autoinflammatory disease caused by pathogenic gain-of-function variants in the gene encoding alpha-kinase 1 (ALPK1) resulting in inappropriate kinase signaling and activation of downstream pro-inflammatory responses mediated by the TIFA/TRAF6/NF-κB pathway.

The most common presenting symptom is a progressive decline in visual acuity that typically begins before 20 years of age, with ophthalmologic examination often revealing optic disc elevation, uveitis, and retinal nerve degeneration. ROSAH patients also exhibit inflammatory features such as non-infectious low-grade fevers, arthralgia, headaches, and persistently elevated levels of serum inflammatory cytokines including tumor necrosis factor α (TNFα), interleukin 6 (IL-6), and IL-1β. ROSAH has been shown to be caused by at least two heterozygous missense variants in the gene encoding alpha-kinase 1 (ALPKI), p.Thr237Met (T237M) and p.Tyr254Cys (Y254C), the T237M gain-of-function mutation being the most frequently reported.

ALPK1 is a member of the atypical human alpha-kinase family and functions as an innate immune pattern recognition receptor (PRR) for ADP-heptose (ADPH), a soluble byproduct of the metabolic processing of bacterial lipopolysaccharide (LPS). ADPH sensing by ALPK1 triggers its phosphorylation and the direct ALPK1-mediated phosphorylation of multiple threonine residues in the adaptor protein TIFA. TIFA, in turn, oligomerizes to form TIFAsomes that trigger TRAF6 multimerization and the activation of the proinflammatory transcription factor NF-κB. Consistent with its role as a PRR, ALPK1-dependent innate immunity has been observed for several bacterial infections, and the small molecule-mediated agonism of ALPK1 signaling can protect against hepatitis B virus (HBV). Both the p.T237M and p.Y254C ALPK1 mutations increase basal NF-κB activity in reporter cells, and the former also enhances ALPK1 autophosphorylation and TIFA phosphorylation. Recently, Snelling et al. reported that the p.T237M variant expands the range of ligands to which the mutant enzyme is responsive such that it can be activated by endogenous nucleotide sugars including uridine diphosphate (UDP)-mannose that fail to activate wild-type ALPK1. Gain-of-function mutations in ALPK1 give rise to the inflammatory phenotypes exhibited by individuals with ROSAH syndrome.

At present, there is no approved drug for the treatment of ROSAH patients. Drugs that antagonize cytokines (IL-6, TNF-α, IL-1β) including tocilizumab, adalimumab, anakinra, and canakinumab have all been given off-label to ROSAH patients with reports of varying levels of efficacy.

### BRIEF SUMMARY OF THE INVENTION

The present invention discloses a method for treating ROSAH syndrome with a structurally novel small molecule ALPK1 inhibitor, which has excellent pharmacokinetic properties. The ALPK1 inhibitor has the potential to serve as a precision-targeted drug for the treatment of ROSAH syndrome.

The present invention relates to a method for treating ROSAH syndrome in a subject in need thereof, comprising administering a pharmaceutically effective amount of a compound of formula (I) or a pharmaceutically acceptable salt thereof,

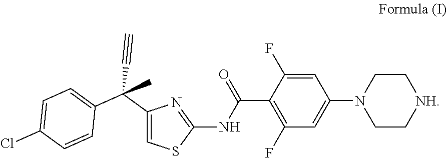 [SMILES: C#C[C@@](C)(c1ccc(Cl)cc1)c1csc(NC(=O)c2c(F)cc(N3CCNCC3)cc2F)n1]

In some embodiments, the compound of formula (I) is administered orally.

In some embodiments, the subject is a mouse or a ROSAH patient.

In some embodiments, the compound of formula (I) is administered at 2-10 mg/kg, once per day.

In some embodiments, the compound of formula (I) is administered at 2, 3, 5 or 10 mg/kg, once per day.

In some embodiments, the compound of formula (I) is administered for 5-10 days.

In some embodiments, the compound of formula (I) crosses the blood-retina barrier, or blood-brain barrier.

In some embodiments, the method is safe without any systemic or ocular complications.

In some embodiments, the compound of formula (I) is administered at 3 or 5 mg/kg once per day for 10 days, maximum plasma concentrations (Cₘₐₓ) of the compound of formula (I) ranges from 160 and 310 ng/mL after 10^{th} daily oral dosing.

In some embodiments, the compound of formula (I) is administered at 3 or 5 mg/kg once per day for 10 days, an average time to Cₘₐₓ (Tₘₐₓ) ranges from 2-2.67 h after 10^{th} daily oral dosing.

In some embodiments, the compound of formula (I) is administered at 3 or 5 mg/kg once per day for 10 days, measured half-life (t_{1/2}) values for the compound of formula (I) in the 3 and 5 mg/kg groups following the 10^{th} dose is 7.69 and 8.81 h, respectively.

The present invention relates to a method for inhibiting ALPK1, comprising administering a pharmaceutically effective amount of a compound of formula (I) or a pharmaceutically acceptable salt thereof,

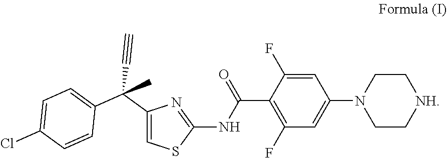 [SMILES: C#C[C@@](C)(c1ccc(Cl)cc1)c1csc(NC(=O)c2c(F)cc(N3CCNCC3)cc2F)n1]

In some embodiments, the method is conducted in vitro, ex vivo, or in vivo.

In some embodiments, the ALPK1 is activated by an ALPK1 agonist, such as ADPH or D-glycero-D-manno-6-fluoro-heptose-1β-S-ADP (DF-006).

In some embodiments, the ALPK1 is inhibited with an IC₅₀ of 0.75-4 nM, such as 1.5 nM.

In some embodiments, the IC₅₀ is measured by measuring the remaining radioactive phosphorylated substrate to quantify ALPK1 kinase activity, which is expressed as the percentage of remaining ALPK1 kinase activity relative to vehicle (DMSO, without the compound of formula (I)) conditions.

In some embodiments, the ALPK1 is inhibited with 400-1600 fold, such as 860-fold selectivity over TAOK2/TAO1.

In some embodiments, the ALPK1 is inhibited with 1000-4000 fold, such as 2067-fold selectivity over CAMK2g.

In some embodiments, the ALPK1 is inhibited with 1500-6000 fold, such as 3067-fold selectivity over CAMK1a.

The present invention relates to a method for reducing the levels of inflammatory cytokine and chemokine medicated by ALPK1, comprising administering a pharmaceutically effective amount of a compound of formula (I) or a pharmaceutically acceptable salt thereof,

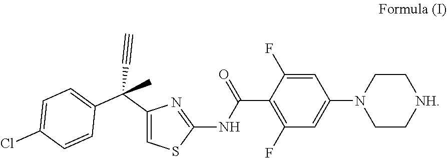 [SMILES: C#C[C@@](C)(c1ccc(Cl)cc1)c1csc(NC(=O)c2c(F)cc(N3CCNCC3)cc2F)n1]

In some embodiments, the method is conducted in vitro, ex vivo, or in vivo.

In some embodiments, the inflammatory cytokine and chemokine is selected from TNF and CXCL8; preferably, the inflammatory cytokine and chemokine is activated by an ALPK1 agonist, such as ADPH or D-glycero-D-manno-6-fluoro-heptose-1β-S-ADP (DF-006).

In some embodiments, when the method is conducted in vitro, the method is conducted in cells, such as immune cells (e.g. THP-1 macrophages), HEK-293 cells stably overexpressing ALPK1 or NF-κB reporter cells overexpressing ALPK1; preferably, the cells are triggered by an ALPK1 agonist, such as ADPH or D-glycero-D-manno-6-fluoro-heptose-1β-S-ADP (DF-006).

In some embodiments, when the inflammatory cytokine and chemokine is TNF in THP-1 macrophages triggered by DF-006, the TNF upregulation is inhibited with an IC₅₀ of 4-15 nM, such as 8 nM, in the form of mRNA fold change.

In some embodiments, when the inflammatory cytokine and chemokine is CXCL8 in THP-1 macrophages triggered by DF-006, the CXCL8 upregulation is inhibited with an IC₅₀ of 3-12 nM, such as 6.5 nM, in the form of mRNA fold change.

The present invention relates to a method for inhibiting ALPK1^{[T237M]}, comprising administering a pharmaceutically effective amount of a compound of formula (I) or a pharmaceutically acceptable salt thereof,

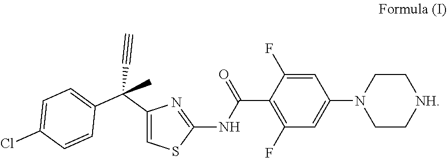 [SMILES: C#C[C@@](C)(c1ccc(Cl)cc1)c1csc(NC(=O)c2c(F)cc(N3CCNCC3)cc2F)n1]

In some embodiments, the method is conducted in vitro, ex vivo, or in vivo.

In some embodiments, the ALPK1^{[T237M]} is activated by ADPH or UDP-mannose.

In some embodiments, UDP-mannose-stimulated ALPK1^{[T237M]} is inhibited in a dose-dependent manner with an IC₅₀ of 8-30 nM, such as 16 nM.

In some embodiments, the IC₅₀ is measured by measuring the remaining radioactive phosphorylated substrate to quantify ALPK1^{[T237M]} kinase activity, which is expressed as the percentage of remaining ALPK1^{[T237M]} kinase activity relative to vehicle (DMSO, without the compound of formula (I)) conditions.

The present invention relates to a method for reducing the levels of inflammatory cytokine and chemokine medicated by ALPK1^{[T237M]}, comprising administering a pharmaceutically effective amount of a compound of formula (I) or a pharmaceutically acceptable salt thereof,

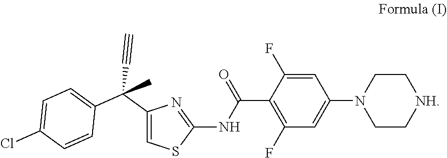 [SMILES: C#C[C@@](C)(c1ccc(Cl)cc1)c1csc(NC(=O)c2c(F)cc(N3CCNCC3)cc2F)n1]

In some embodiments, the method is conducted in vitro, ex vivo, or in vivo.

In some embodiments, when the method is conducted in vitro, the method is conducted in cells, such as ALPK1^{[T237M]} overexpression cells (e.g. HEK-293 cells stably overexpressing ALPK1^{[T237M]} or NF-κB reporter cells overexpressing ALPK1^{[T237M]}).

In some embodiments, in NF-κB reporter cells overexpressing ALPK1^{[T237M]}, NF-κB reporter activity is enhanced in the absence of exogenous stimulation, and NF-κB reporter activity is inhibited in a dose-dependent manner by the compound of formula (I).

In some embodiments, in HEK-293 cells stably overexpressing ALPK1^{[T237M]}, TNF, CXCL10, and CXCL8 expressions are increased in the absence of exogenous stimulation, and TNF, CXCL10, and CXCL8 expressions are inhibited in a dose-dependent manner by the compound of formula (I).

In some embodiments, in HEK-293 cells stably overexpressing ALPK1^{[T237M]}, CXCL10 is inhibited with an IC₅₀ of 70-260 nM, such as 134 nM, in the form of mRNA fold change.

In some embodiments, in HEK-293 cells stably overexpressing ALPK1^{[T237M]}, TNF is inhibited with an IC₅₀ of 45-180 nM, such as 92 nM, in the form of mRNA fold change.

In some embodiments, in HEK-293 cells stably overexpressing ALPK1^{[T237M]}, CXCL8 is inhibited with an IC₅₀ of 70-260 nM, such as 136 nM, in the form of mRNA fold change.

In some embodiments, the enhanced secretion of CXCL8 from HEK-293 cells overexpressing ALPK1^{T237M} is inhibited with an IC₅₀ of 60-250 nM, such as 125 nM.

In some embodiments, the inflammatory cytokine and chemokine is selected from TNF, CXCL10, and CXCL8; or, the inflammatory cytokine and chemokine is selected from Ccl2, Ccl5, Cxcl1, Cxcl9, Cxcl10, Tnf, Il6, Cx3cr1 and Aif1.

In some embodiments, the inflammatory cytokine and chemokine is upregulated by UDP-mannose.

In some embodiments, when the method is conducted in vivo, a subject is a mouse model of ROSAH syndrome; such as a mouse heterozygous for the hALPK1 and hALPK1^{T237M} alleles.

In some embodiments, when the method is conducted in vivo, a subject is a ROSAH patient.

In some embodiments, the compound of formula (I) is administered orally.

In some embodiments, the compound of formula (I) is administered at 2-10 mg/kg, once per day.

In some embodiments, the compound of formula (I) is administered at 2, 3, 5 or 10 mg/kg, once per day.

In some embodiments, the compound of formula (I) is administered for 5-10 days.

In some embodiments, when the method is conducted in vivo, Cxcl10, Ccl2, and Ccl5 expressions in retina in ROSAH model mice are inhibited by 30-50%, such as 31%, 40%, and 50%, respectively, in the form of mRNA fold change, with the compound of formula (I) once per day for 10 days at the dose of 5 mg/kg.

In some embodiments, Cxcl10, Ccl2, and Ccl5 expressions in retina are upregulated by 305-, 102-, and 102-fold in ROSAH model mice in the form of mRNA fold change, and their induction are inhibited by the compound of formula (I) (once per day for 10 days at the dose of 5 mg/kg) by 30-50%, such as 31%, 40%, and 50%, respectively.

In some embodiments, when the method is conducted in vivo, Ccl2 and Cxcl10 expressions in optical nerves in ROSAH model mice are inhibited by 60-95%, such as 66% and 91%, respectively, in the form of mRNA fold change, with the compound of formula (I) once per day for 10 days at the dose of 5 mg/kg.

In some embodiments, Ccl2 and Cxcl10 expressions in optical nerves are upregulated by 4.5- and 41-fold in ROSAH model mice in the form of mRNA fold change, and their induction are inhibited by the compound of formula (I) (once per day for 10 days at the dose of 5 mg/kg) by 60-95%, such as 66% and 91%, respectively.

In some embodiment, when the method is conducted in vivo, Ccl2, Ccl5, Cxcl1, and Cxcl10 expressions in cortical samples from the brains in ROSAH model mice are inhibited by 45-75%, such as 71%, 56%, 49% and 55%, respectively, in the form of mRNA fold change, with the compound of formula (I) once per day for 10 days at the dose of 5 mg/kg.

In some embodiments, Ccl2, Ccl5, Cxcl1, and Cxcl10 expressions in cortical samples from the brains are upregulated by 15-, 4.4-, 15-, and 196-fold in ROSAH model mice in the form of mRNA fold change, and their induction are inhibited by the compound of formula (I) (once per day for 10 days at the dose of 5 mg/kg) by 45-75%, such as 71%, 56%, 49% and 55%, respectively.

In some embodiments, CXCL8 induction is inhibited in a dose-dependent manner in ROSAH patient whole blood with an IC₅₀ of 20-80 nM, such as 42 nM; preferably, CXCL8 is induced by acetylated UDP-mannose.

The present invention relates to a method for inhibiting microglia or astrocyte activation in a subject in need thereof, comprising administering a pharmaceutically effective amount of a compound of formula (I) or a pharmaceutically acceptable salt thereof,

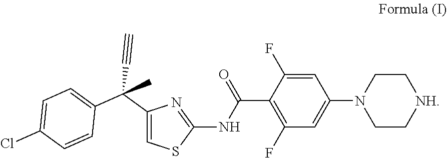 [SMILES: C#C[C@@](C)(c1ccc(Cl)cc1)c1csc(NC(=O)c2c(F)cc(N3CCNCC3)cc2F)n1]

In some embodiments, the compound of formula (I) is administered orally.

In some embodiments, the compound of formula (I) is administered at 2-10 mg/kg, once per day.

In some embodiments, the compound of formula (I) is administered at 2, 3, 5 or 10 mg/kg, once per day.

In some embodiments, the compound of formula (I) is administered for 5-10 days.

In some embodiments, the subject is a mouse model of ROSAH syndrome; such as a mouse heterozygous for the hALPK1 and hALPK1^{T237M} alleles.

In some embodiments, the microglia is the microglia (Iba1⁺ cells) in both the outer nuclear layer (ONL) and inner nuclear layer (INL) of the retina. In some embodiments, the astrocyte is the astrocyte in retina nerve fiber layer.

In some embodiments, microglial activation in the INL and ONL are inhibited by 50-70% in ROSAH model mice, such as 68% and 53%, respectively, with the compound of formula (I) once per day for 10 days at the dose of 3 mg/kg.

In some embodiments, astrocyte activity is inhibited by 35%-60%, such as 49% in ROSAH model mice, with the compound of formula (I) once per day for 10 days at the dose of 3 mg/kg.

### BRIEF DESCRIPTION OF THE DRAWINGS

FIG. 1 is changes of ALPK1 kinase activity after adding DF-003, which is expressed as the percentage of remaining ALPK1 kinase activity relative to vehicle (DMSO, no DF-003) conditions.

FIG. 2 is inhibition of DF-003 on TNF upregulation induced by DF-006 in THP-1

macrophages.

FIG. 3 is inhibition of DF-003 on CXCL8 upregulation induced by DF-006 in THP-1 macrophages.

FIG. 4 is TIFA phosphorylation by ALPK1^{[T237M]} in the absence or presence of exogenous activating ligands (ADPH and UDP-mannose).

FIG. 5 is changes of UDP-mannose-stimulated ALPK1^{[T237M]} kinase activity after adding DF-003.

FIG. 6 is changes of NF-κB activity after adding DF-003 in NF-κB reporter cells overexpressing human ALPK1 or ALPK1^{[T237M]}.

FIG. 7 is fold induction of CXCL10, TNF, and CXCL8 mRNA levels in vehicle (DMSO)-treated ALPK1^{[T237M]} OE cells compared to those in ALPK1 OE cells.

FIG. 8 is inhibition of CXCL10 mRNA upregulation in ALPK1 OE and ALPK1^{[T237M]} OE cells treated with DF-003.

FIG. 9 is inhibition of INF mRNA upregulation in ALPK1 OE and ALPK1^{[T237M]} OE cells treated with DF-003.

FIG. 10 is inhibition of CXCL8 mRNA upregulation in ALPK1 OE and ALPK1^{[T237M]} OE cells treated with DF-003.

FIG. 11 is supernatant CXCL8 levels in HEK-293 cells overexpressing ALPK1^{T237M} after adding DF-003.

FIG. 12 is DF-003 concentrations in the plasma at various time points through 24 h after the last (10^{th}) dose.

FIG. 13 is DF-003 concentrations in tissues at 24 h after the last (10^{th}) dose.

FIG. 14 is a diagram of preparing hALPK1-KI and hALPK1^{[T237M]}-KI mice (ROSAH model mice).

FIG. 15 is retinal cross-sections stained with anti-Iba1 after dosing, wherein microglia is labelled as green.

FIG. 16 is Iba1-positive cells in the inner nuclear layer (INL) of the retina from each mouse are quantified and normalized to the length of the curvature of the retina.

FIG. 17 is Iba1-positive cells in the outer nuclear layer (ONL) of the retina from each mouse are quantified and normalized to the length of the curvature of the retina.

FIG. 18 is retinal cross-sections stained with anti-GFAP after dosing, wherein the activated astrocytes are labelled via immunohistochemistry.

FIG. 19 is the percentage of the retinal length of the nerve fiber layer that was positive for GFAP-labeled astrocyte fibers.

FIG. 20 is mRNA levels of the pro-inflammatory genes in the retina samples from hALPK1-KI and hALPK1^{[T237M]}-KI mice.

FIG. 21 is mRNA levels of the pro-inflammatory genes in the optic nerve samples from hALPK1-KI and hALPK1^{[T237M]}-KI mice.

FIG. 22 is mRNA levels of the pro-inflammatory genes in the brain cortex samples from hALPK1-KI and hALPK1^{[T237M]}-KI mice.

FIG. 23 is the body weight change in hALPK1-KI or hALPK1^{[T237M]}-KI mice after dosing.

FIG. 24 is fold changes of CXCL8 induction by UDP-mannose and inhibition by DF-003 in whole blood from a patient with ROSAH syndrome.

FIG. 25 is fold changes of CXCL8 induction by UDP-mannose and inhibition by DF-003 in whole blood from the control (healthy mother).

### DETAILED DESCRIPTION OF THE PREFERRED EMBODIMENT

The following examples further illustrate the present invention, but the present invention is not limited thereto.

Below presents preferred embodiments of the present invention based on the drawings in order to illustrate the technical schemes of the present invention in detail.

### Structures of DF-003, ADPH and DF-006

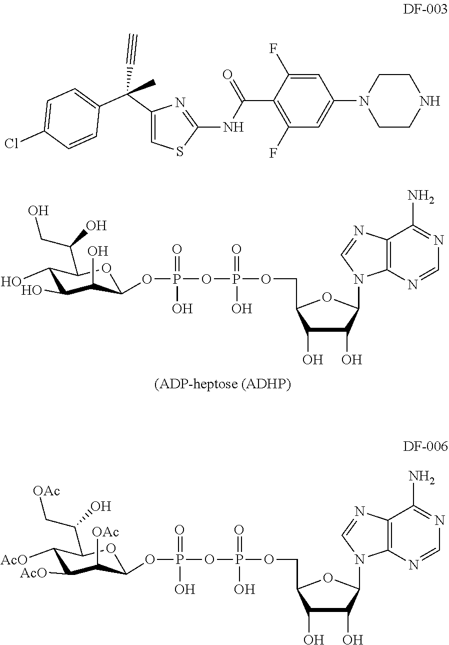

### Sex as a Biological Variable

Both male and female mice were used to conduct the experiments reported herein, and no specific or consistent differences were observed as a function of sex.

### Recombinant Protein Preparation

DNA sequences coding for human ALPK1 (NP_079420.3) and ALPK^{T237M}, with 3×Flag-tag (MDYKDHDGDYKDHDIDYKDDDDK) sequences added to the C-terminus of the protein, were codon optimized for optimal expression in insect cell lines. The DNA sequence was cloned into the pFAST-BAC vector for the generation of insect transducing baculovirus. To produce recombinant human ALPK1 or ALPK1^{[T237M]} proteins, Sf9 cells were transduced with baculovirus. Cells were homogenized in 50 mM Tris, 0.5 M NaCl, 5% glycerol, pH 8.0; proteins were bound to anti-flag affinity beads, washed, and eluted using 3×Flag peptides in the same solution. The coding sequence for human TIFA was synthesized and cloned into the pET28a (+) vector, which was used to transform E. coli. Recombinant His-tagged TIFA was purified with a Ni column and finally dissolved in a buffer containing 50 mM Tris and 150 mM NaCl (pH 8.0).

### Quantitative Real-Time PCR

RNA was extracted from harvested tissue and cell samples using the TRI reagent (T9424, Sigma-Aldrich). The HiScript Q-RT SuperMix (Vazyme, Nanjing, China) was then used to synthesize cDNA. Real-time PCR was carried out using a QuantStudio™ 5 Real-Time PCR System (Applied Biosystems) with the AceQ qPCR SYBR Green Master Mix Kit (Vazyme). All qPCR assays were run in 384-well thin-wall plates with a total reaction volume of 10 μL, including 2 μL of cDNA (20 ng), 5 μL of 2×AceQ qPCR SYBR Green Master Mix, 0.2 μL of ROX Reference dye (50×), 0.1 μL of each primer (F+R, 10 μM of each), and 2.6 μL of ddH₂O. Thermocycler settings included an initial 5 min at 95°° C. followed by 40 cycles of 95° C. for 10 s and 60° C. for 30 s. Relative gene expression was analyzed using the 2^{-ΔΔCt} method, and GAPDH served as a normalization control for human cell lines and Rpl13 for murine tissues. Data are presented as the fold-change in expression relative to vehicle control unless otherwise noted. Primers used for these analyses are presented in the following Table 1.

TABLE 1
qPCR primers used in this study

| | |
|---|---|
| Gene name | Sequence |

HUMAN

| | |
|---|---|
| TNF | CTCTTCTGCCTGCTGCACTTTG |
| | ATGGGCTACAGGCTTGTCACTC |
| CXCL10 | GGTGAGAAGAGATGTCTGAATC |
| | CGTCCATCCTTGGAAGCACTGC |
| | A |
| CXCL8 | AAGGTGCAGTTTTGCCAAGGCC |
| | CAGTTTTCCTTGGGGTCC |
| GAPDH | AATTCCATGGCACCGTCAAGTG |
| | GACTCCACGACGTACTCA |

MOUSE

| | |
|---|---|
| Ccl2 | CCAATGAGTAGGCTGGAGAGCG |
| | ACCCATTCCTTCTTGGGGTC |
| Ccl5 | CTCACCATATGGCTCGGACACG |
| | ACTGCAAGATTGGAGCAC |
| Cxcll | ACTCAAGAATGGTCGCGAGGGT |
| | GCCATCAGAGCAGTCTGT |
| Cxcl9 | CCTAGTGATAAGGAATGCACGA |
| | TGCTAGGCAGGTTTGATCTCCG |
| | TTC |
| Cxcl10 | CCACGTGTTGAGATCATTGCCG |
| | AGGCTCTCTGCTGTCCATC |
| Cx3cr1 | GTGAGTGACTGGCACTTCCTGA |
| | CCGAACGTGAAGACGAGG |
| Aif1 | TCTGCCGTCCAAACTTGAAGCC |
| | CTCTTCAGCTCTAGGTGGGTCT |
| Tnf | CCCTCACACTCAGATCATCTTC |
| | TGCTACGACGTGGGCTACAG |
| Rpl13 | CTGCTCTCAAGGTTGTTCGGCT |
| | CCTTCCGTTTCTCCTCCAGAGT |

### Example 1

### In Vitro Radiolabeled Kinase Assays (ALPK1)

### a) Experiment Method

In vitro analyses of DF-003 IC₅₀ values for ALPK1 were performed by Reaction Biology (Malvern, PA, USA). Recombinant human ALPK1 (0.5 nM), human TIFA (10 μM), and ADP-heptose (5 nM) were mixed in a kinase reaction buffer (20 mM HEPES (pH 7.5), 10 mM MgCl₂, 1 mM EGTA, 0.01% Brij35, 0.02 mg/mL BSA, 0.1 mM Na₃VO₄, 2 mM DTT, 1% DMSO) to which 3-fold dilutions of DF-003 (1 μM-50.8 pM; prepared in DMSO) were added. After the addition of [³³P]-ATP (20 μM; specific activity: 0.01 μCi/μL final), the kinase reaction was allowed to proceed at room temperature for 60 min, and samples were then spotted onto P81 ion exchange paper (#3698-915, Whatman). After extensive washing with 0.75% phosphoric acid, the radioactive phosphorylated substrate remaining on the filter paper was measured to quantify ALPK1 kinase activity, which was expressed as the percentage of remaining ALPK1 kinase activity relative to vehicle (DMSO, no DF-003) conditions. Curve fitting and IC₅₀ value calculations were performed using GraphPad Prism 4.

### 2. Result

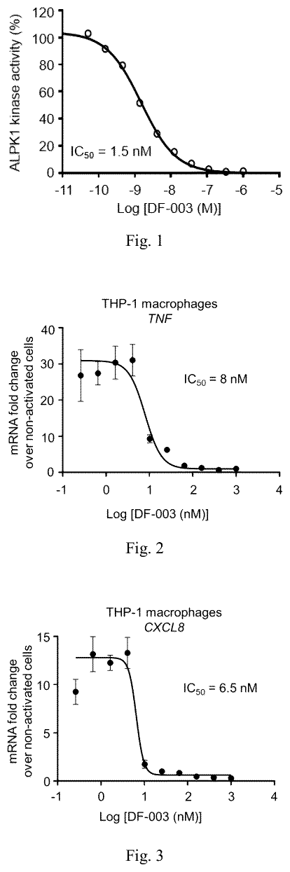

DF-003 dose-dependently inhibited ALPK1 kinase activity with an IC₅₀ of 1.5 nM (FIG. 1).

### Example 2

### HotSpot Kinase Assay

### 1. Experiment Method

The inhibitory effects of DF-003 on the activity of 394 human kinases were measured by Reaction Biology using the previously described HotSpot™ Kinase Assay (Reference: Anastassiadis T, et al. Comprehensive assay of kinase catalytic activity reveals features of kinase inhibitor selectivity. Nat Biotechnol. 2011; 29(11):1039-45.). The general experimental approach was the same as the in vitro kinase assay detailed above, with an extended 120-minute reaction time, an ATP concentration of 10 μM, and a single tested DF-003 dose (10 μM). The percentage of kinase activity was calculated for each kinase with DMSO and DF-003 treatment. The dose-dependent inhibitory activity of DF-003 against the top 3 non-ALPK1 human kinases that were most strongly inhibited by 10 μM DF-003 was further determined in kinase reaction assays using a 10-dose dilution series of DF-003 (20 μM-0.763 nM), with all other experimental conditions being the same as above. Additionally, a 10-dose dilution series of staurosporine and other enzyme-appropriate compounds were included as positive control inhibitors.

### 2. Result

DF-003 exhibited excellent selectivity for ALPK1 when tested against a panel of 394 human kinases. The IC₅₀ values for the three non-ALPK1 kinases against which DF-003 exhibited the highest levels of activity were 1.29 μM, 3.10 μM, and 4.60 μM for TAOK2/TAO1, CAMK2g, and CAMK1a, respectively. Therefore, DF-003 is a potent and selective inhibitor of ALPK1.

### Example 3

### Inhibition of Chemokine and Cytokine Production in Response to ALPK1 Activation

ADPH is well-established as a canonical pathogen-associated molecular pattern (PAMP) that triggers ALPK1 activation, serving as an essential mediator for the upregulation of inflammatory cytokines and chemokines including TNFα and CXCL8 (also known as IL-8) in response to bacterial infection. We previously developed the ADPH derivative DF-006 as a serum-stable small molecule ALPK1 agonist that triggers TIFA phosphorylation and upregulation of Tnf and other proinflammatory genes. We tested the ability of DF-003 to suppress the induction of inflammatory cytokine and chemokine gene expression in THP-1 macrophages triggered by DF-006.

### 1. Experiment Method

THP-1 cells (Cell Bank of The Chinese Academy of Sciences, Beijing, China) were routinely cultured in RPMI-1640 (Hyclone, Logan, UT, USA) containing 10% heat-inactivated fetal bovine serum (FBS, Hyclone), 0.05 mM 2-mercaptoethanol, 1% penicillin (100 U/mL), and streptomycin (100 μg/mL) (Gibco, Waltham, MA, USA) in a humidified 5% CO₂ incubator at 37° C. To induce macrophage-like differentiation, these cells were seeded in 24-well plates (4×10⁵/well) and treated for 48 h with phorbol myristate acetate (PMA; 50 ng/ml). Media was then exchanged for fresh complete medium without PMA and pretreated with a range of DF-003 concentrations for 2 h (0.3 nM-1000 nM), after which they were stimulated for an additional 4 h using 5 nM D-glycero-D-manno-6-fluoro-heptose-1β-S-ADP (DF-006). Cells were then collected for qPCR to analyze the expression of TNF and CXCL8 (See above), with GAPDH serving as a normalization control. Data were expressed as mRNA fold induction compared to THP-1 macrophages not treated with DF-006 activation. Samples were analyzed in quadruplicate, and IC₅₀ values for the inhibition of ALPK1 agonist-induced cytokine upregulation were calculated by plotting relative mRNA expression against DF-003 concentration for each gene of interest using GraphPad Prism 6.

### 2. Result

DF-003 exhibited IC₅₀ values of 8 nM and 6.5 nM for the inhibition of DF-006-induced TNF and CXCL8 upregulation, respectively (FIGS. 2-3, wherein data represent means±SEM for the quadruplicate wells at each treatment dose.). These data demonstrate that DF-003 disrupts inflammatory signaling downstream of ligand-mediated ALPK1 activation in immune cells.

### Example 4

### Inhibition of TIFA Phosphorylation Mediated by ALPK1^{T237M}

To assess whether DF-003 was also able to inhibit the ROSAH-associated ALPK1^{[T237M]} mutated enzyme, we performed an in vitro kinase assay with recombinant human TIFA and ALPK1^{[T237M]} in the presence or absence of ADPH or UDP-mannose, a reported endogenous ligand for ALPK1^{[T237M]}.

1. Experiment mMthod

The general experimental approach was almost the same as the in vitro kinase assay mentioned in example 1, except that recombinant human ALPK1^{[T237M]} was contained in a kinase reaction buffer instead of the recombinant human ALPK1, and ADP-heptose (5 nM) or UDP-mannose (10 μM) is used.

### 2. Result

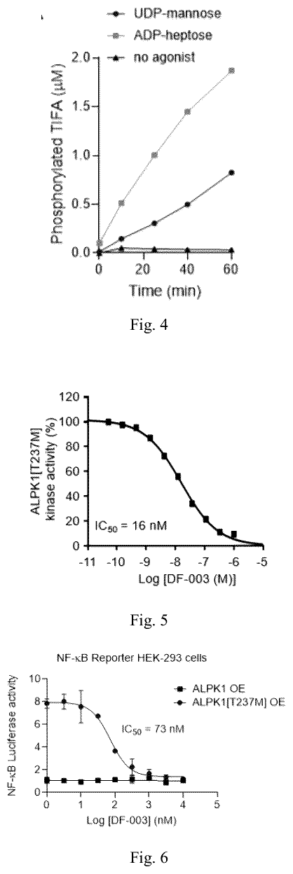

We observed that ALPK1^{[T237M]} was inactive in the absence of these exogenous activating ligands, whereas it phosphorylated TIFA in a time-dependent manner in response to both ADPH and UDP-mannose (FIG. 4). DF-003 inhibited UDP-mannose-stimulated ALPK1^{[T237M]} in a dose-dependent manner with an IC₅₀ of 16 nM (FIG. 5). DF-003 is thus able to potently inhibit ALPK1^{[T237M]}-induced TIFA phosphorylation.

### Example 5

### Inhibition of Downstream Inflammatory Cytokine Production Mediated by ALPK1^{T237M}

To assess whether DF-003 could inhibit ALPK1^{[T237M]} and associated downstream signaling pathways in human cells. To that end, we established NF-κB reporter cells overexpressing (OE) ALPK1 or ALPK1^{[T237M]}.

### 1. Experiment Method

HEK-293 cells were purchased from the Cell Bank of The Chinese Academy of Sciences and routinely cultured in DMEM/High-Glucose (Hyclone). To obtain HEK-293 cells stably overexpressing ALPK1 or ALPK1^{[T237M]}, codon-optimized DNA coding sequencing for Flag-tagged human ALPK1 and ALPK1^{T237M} were inserted into the pcDNA3.1 vector. Plasmids were transfected into HEK-293 cells using Lipofectamine 2000 as per the manufacturer's protocol. At 48 h post-transfection, G418 (#A1720, Sigma-Aldrich, St. Louis, MO, USA) was used to select resistant transformed cells, and single-cell clones were prepared through a limiting dilution-based approach. Western blotting was used to assess Flag-tagged ALPK1 and ALPK1^{T237M} transgene overexpression. Cells were treated in triplicate with a range of DF-003 concentrations (0.64 nM-20 μM) for a total of 30 h, during which media was refreshed with equivalent DF-003 doses after 24 h. Cells were then harvested to analyze the expression of CXCL10, TNF, and CXCL8 by qPCR (See above), and CXCL10, TNF, and CXCL8 mRNA levels were normalized to levels of GAPDH. Data are expressed as fold induction of mRNA levels compared to vehicle-treated ALPK1 OE cells. CXCL8 concentrations in supernatants collected from these cells were analyzed with a Human CXCL8 ELISA Set (#555244, BD Biosciences, Franklin Lakes, NJ, USA) based on the manufacturer's instructions, and results were analyzed with a microplate reader (Multiskan FC, Thermo Fisher Scientific).

To prepare stable NF-κB reporter cells, HEK-293 cells were transfected with an NF-κB dual reporter construct (a kind gift from Dr. Lei Sun of Fudan University). The construct contains an NF-κB responsive element (5′-GGGAATTTCCGGGAATTTCCGGGAATTTCCGGGAATTTCCGGGAATTTCCGGGAATT TCCGGGAATTTCCGGGAATTTCC-3′), a minimized CMV promoter, followed by coding sequences for secreted alkaline phosphatase (SEAP) and firefly luciferase linked by a 2A peptide. G418 (#A1720, Sigma-Aldrich) was used to select resistant transformed cells, and single-cell clones were prepared through a limiting dilution-based approach. Positive clones were confirmed by increased alkaline phosphatase activity and luciferase activity after stimulating cells with DF-006. To measure increased NF-κB signal caused by ALPK1^{[T237M]} and DF-003's inhibitory effect, reporter cell clones were transfected with ALPK1 or ALPK1^{[T237M]} expression vectors. Four hours after transfection, cells were treated with serially diluted DF-003 for an additional 48 hours. Intracellular luciferase activity was measured using Luciferase Reporter Gene Assay Kit (Beyotime, Jiangsu, China; RG027) per the manufacturer's instructions.

### 2. Result

We observed enhanced NF-κB reporter activity in the absence of exogenous stimulation in the ALPK1^{[T237M]} OE cells, and DF-003 treatment suppressed this activity in a dose-dependent manner (FIG. 6).

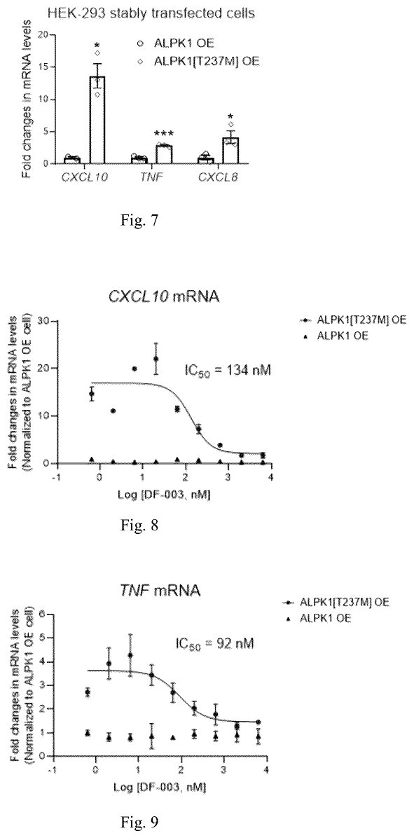

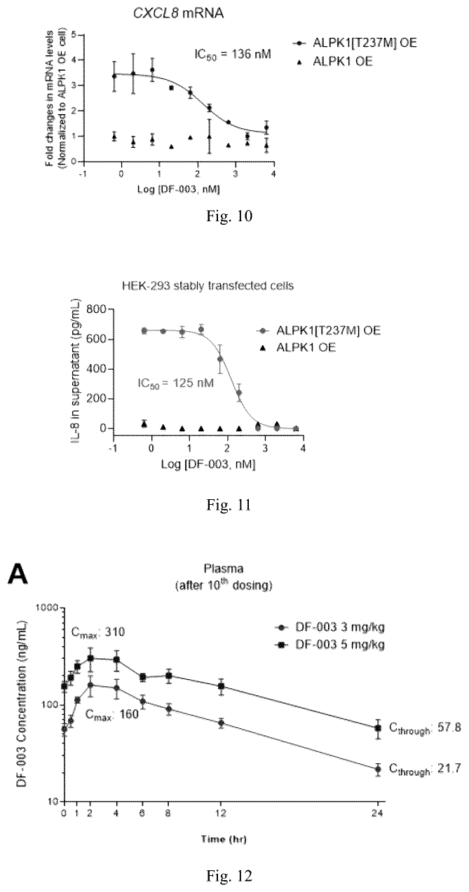

In HEK-293 cells, overexpression of ALPK1^{[T237M]} increased TNF, CXCL10, and CXCL8 expression relative to the levels of these mRNAs in cells overexpressing wild-type ALPK1 (FIG. 7). DF-003 dose-dependently inhibited cytokine upregulation in HEK-293 cells overexpressing ALPK1^{[T237M]} and had no impact on the basal expression of TNF, CXCL10, or CXCL8 in cells overexpressing wild-type ALPK1 (FIGS. 8-10). Consistent with these changes at the mRNA level, DF-003 suppressed (IC₅₀=125 nM) the enhanced secretion of CXCL8 from HEK-293 cells overexpressing ALPK1^{T237M} (FIG. 11). DF-003 is thus able to potently inhibit ALPK1^{[T237M]}-induced downstream inflammatory cytokine and chemokine production in human cell lines. In FIGS. 7-11, data represent the mean±SEM for the triplicate wells of each treatment group. In FIGS. 8-10, statistical comparisons to the wild-type ALPK1-overexpressing stable cell line group were made using the two-tailed unpaired Student's t-tests: *in p<0.05, ***p<0.001.

### Example 6

### Evaluation of the Pharmacokinetic Properties of DF-003

To support the in vivo use of DF-003 for ROSAH syndrome, we analyzed its pharmacokinetic properties in wild-type C57BL/6J mice.

### 1. Experiment Method

Six 8-week-old male C57BL/6J mice from Zhejiang Vital River Laboratory Animal Technology Co., Ltd. (Zhejiang, China) were randomly assigned to 2 groups (3 animals/group). Animals were administered DF-003 by oral gavage once daily at 3 and 5 mg/kg for 10 consecutive days. DF-003 were dissolved in 0.1% (w/v) methyl cellulose (MC, 1500cP)+0.2% (v/v) Tween 80 in purified water. The oral dosing volume for all animals was 10 mL/kg body weight. Drinking water and certified rodent diet were available to animals ad libitum, except that animals were fasted 16 h prior to the last administration until 4 h after the last dosing. Blood samples at 0 (right before the last dosing), 0.5, 1, 2, 4, 6, 8, 12, and 24 h after the 10^{th} dosing were collected from the tail vein and transferred into tubes containing K₂-EDTA. The tubes were gently inverted several times to ensure mixing and immediately placed on wet ice. Plasma was obtained by centrifugation at 3200×g at 4° C. for 10 min and stored at ≤−60° C. until subsequent analysis. Twenty-four hours after the 10^{th} dosing, the animals were euthanized by CO₂ inhalation. The eyes were harvested, and the retinas and optic nerves were separated. In addition, a piece of cerebral cortex tissue (˜50 μg) from each mouse was snap-frozen in liquid nitrogen. The concentrations of DF-003 in the plasma, retina, and cerebral cortex of each mouse were measured by Wuhan Haipu Biomed Inc. (Wuhan, China) using an LC-MS/MS approach. The concentration of DF-003 in the optic nerve was also measured using this same strategy, combining all 6 optic nerves from the 3 mice in each dosing group as a single sample.

### 2. Result

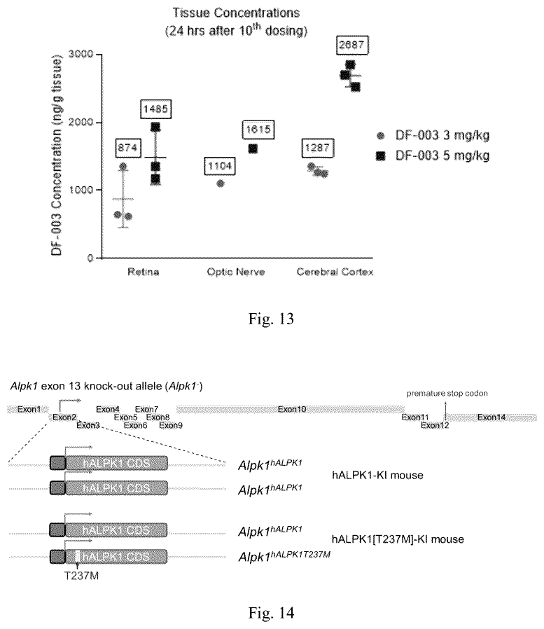

Oral administration of DF-003 at 3 or 5 mg/kg once per day for 10 days was associated with a dose-dependent increase in plasma drug concentrations at all time points from 0-24 h post the last (10^{th}) dosing (FIG. 12). Maximum plasma concentrations (Cₘₐₓ) of DF-003 rose approximately proportionally with administered dose to 160 and 310 ng/mL in the respective treatment groups. DF-003 had an average time to Cₘₐₓ (Tₘₐₓ) ranging from 2-2.67 h following the 10^{th} dose (Table 2). The measured half-life (t_{1/2}) values for DF-003 in the 3 and 5 mg/kg groups following the 10^{th} dose were 7.69 and 8.81 h, respectively. Orally administered DF-003 also led to a dose-dependent increase in the retinal and optic nerve concentrations after the 10^{th} dose (FIG. 13), indicative of a drug that crosses the blood-retina barrier. High concentrations of DF-003 were also detectable in the cerebral cortex of mice from both dosing groups after the 10^{th} dose, indicative of a drug that also crosses the blood-brain barrier (FIG. 13).

TABLE 2
Pharmacokinetic parameters of DF-003 after 10^{th} daily
oral dosing in male C57BL/6J mice (Data are means ±
SD (n = 3 mice per group)).

| | 3 mg/kg | 5 mg/kg |
|---|---|---|
| Cₘₐₓ (ng/mL) | 160 ± 38.6 | 310 ± 73.5 |
| Tₘₐₓ (h) | 2.00 ± 0.00 | 2.67 ± 1.15 |
| t_{1/2} (h) | 7.69 ± 0.99 | 8.81 ± 1.03 |
| CL/F | 27.6 ± 1.94 | 21.6 ± 4.11 |
| (mL/min/kg) | | |
| AUCO₀₋ₜₐᵤ | 1815 ± 132.0 | 3944 ± 697.0 |
| (h*ng/mL) | | |
| Vz/F (L/kg) | 18.3 ± 1.55 | 16.5 ± 3.68 |
| C_{avg} (ng/mL) | 75.6 ± 5.53 | 164 ± 29.0 |

### Example 7

### Construction of Mouse Model of ROSAH Syndrome and In Vivo Efficacy Studies

### 1. Experiment Methods:

### (1) Mouse Model of ROSAH Syndrome

In an effort to establish a model of ROSAH syndrome, we utilized Alpk1^{-/-} mice generated previously in which the kinase function of murine Alpk1 had been disrupted by exon 13 deletion (reference: Xu C, et al. Alpha-kinase 1 (ALPK1) agonist DF-006 demonstrates potent efficacy in mouse and primary human hepatocyte (PHH) models of hepatitis B. Hepatology. 2023.). CRISPR and homologous recombination were used to introduce the coding sequence of human ALPK1 (hALPK1) or hALPK1^{[T237M]} immediately following the start codon in the murine Alpk1⁻0 allele, with the latter containing the same C>T nucleotide substitution as in human patients bearing the T237M disease-causing mutation. The detailed method is shown as follows:

To prepare ROSAH model mice, a guide RNA (gRNA) (5′-AGTGAGGACCAGCGGTGCAGAGG-3′) targeting exon 2 of the mouse Alpk1 gene (NCBI Gene ID: 71481) was generated. Targeting recombination vectors containing the following elements were prepared: 1) a 1.5 kb 5′ arm homologous to the genomic sequence upstream of the start codon of mouse Alpk1 (including ATG); 2) the human ALPK1 coding sequence (encoding wild-type ALPK1 for the mAlpk1^{hALPK1} allele or ALPK1^{[T237M]} for the mAlpk1^{hALPK1T237M} allele, the latter of which contains the same C>G nucleotide substitution as in human ROSAH syndrome patients) with a poly-A tail; 3) a 1.4 kb 3′ arm homologous to the genomic sequence downstream of the start codon for mouse Alpk1 (excluding ATG). In vitro transcribed gRNA and Cas9 mRNA, together with targeting recombination vectors, were co-microinjected into C57BL/6N Alpk1^{-/-} mice-derived fertilized eggs. Founders with the hALPK1 CDS inserted into the mouse Alpk1 gene were screened via Southern blotting and further confirmed by PCR and Sanger sequencing and were backcrossed with Alpk1^{-/-} mice.

Mice bearing the abovementioned alleles were intercrossed to generate founder mAlpk1^{hALPK1/hALPK1T237M} mice on the C57BL/6N background (Crb1^{rd8/rd8}) . These mice were backcrossed with wild-type C57BL/6J (Crb1^{+/+}) mice twice to respectively obtain mAlpk1^{+/hALPK1}; Crb1^{+/+} and mAlpk1^{+/hALPK1T237M}; Crb1^{+/+} mice. These lines were intercrossed to generate mAlpk1^{hALPK1/hALPK1}; Crb1^{+/+} (designated as hALPK1-KI) and mAlpk1^{hALPK1/hALPK1T237M}; Crb1^{+/+} (designated as hALPK1^{[T237M]}-KI) mouse lines. Both lines exhibit loss-of-function for mouse Alpk1. Sixteen to seventeen-week-old hALPK1-KI and hALPK1^{[T237M]}-KI littermates from the mating between these two lines were used for testing DF-003 efficacy in vivo. Our mice included the introduction of the wild-type Crb1 gene from C57BL/6J mice to address the rd8 mutation in this gene present in the C57BL/6N subline that can confound efforts to analyze ocular phenotypes. Mice were housed in a specific pathogen-free (SPF) animal facility at Drug Farm (Shanghai, China) in individually ventilated cages on a 12-hour light/dark cycle with a bedding of wood shavings and ad libitum access to rodent chow.

### (2) In Vivo Efficacy Studies

To test the in vivo efficacy of DF-003 in ROSAH model mice, female hALPK1-KI and hALPK1^{[T237M]}-KI mice (16-17 weeks of age; n=24/genotype) were each randomized into vehicle (n=8/genotype), DF-003 3 mg/kg (n=8/genotype), and DF-003 5 mg/kg (n=8/genotype) treatment groups, ensuring that there were no differences in starting body weight among groups. Mice were dosed orally with DF-003 (3 or 5 mg/kg) or vehicle control (0.1% MC) once per day for 10 total doses at a dosage volume of 10 mL/kg body weight. The status and body weights of all mice were monitored daily throughout this study. Twenty-four hours after the final dose, one retina, one optic nerve, and brain cortex samples were harvested from each mouse and were immediately homogenized in the TRI reagent (Sigma-Aldrich) and stored at −80° C. for analyses of gene expression. One eyeball from each mouse was fixed with 4% paraformaldehyde, and the retina was dissected and embedded in O.C.T. compound (Sakura Finnetek, Torrance, CA, USA; 4583) followed by storage at −80° C.

### (3) Histological, Immunofluorescent, and Immunohistochemical Staining

Mouse retina samples were fixed in 4% paraformaldehyde (PFA), mounted in O.C.T embedding compound, and frozen at −20° C. to −80° C., after which they were cut into 10 μm transverse sections near the center of the retina (through the optic papilla).

For immunofluorescent and immunohistochemical labeling, frozen sections were air-dried and then blocked using goat serum diluted in PBS containing 0.1% Triton X-100. The microglia were detected by immunofluorescence using primary rabbit anti-Iba1 (Wako, Richmond, VA, USA; 019-19741) and secondary goat anti-rabbit IgG (H+L) Cross-Adsorbed, Alexa Fluor™ 488 (Invitrogen, Carlsbad, CA, USA; A-11008). Nuclei were counterstained with DAPI (Shanghai Zhenghuang, Shanghai, China; C1005). The astrocyte activation marker GFAP was detected by immunohistochemistry with rabbit anti-GFAP (Abcam; ab68428) and secondary donkey anti-rabbit IgG H&L (HRP) (Abcam; ab6802). ImmPACT® DAB Peroxidase (HRP) Substrate (Vector Labs, Burlingame, CA, USA) was used for staining, followed by diaminobenzidine (DAB) coloration according to routine immunohistochemistry procedures, yielding a brown signal corresponding to GFAP positivity. Cell nuclei were counterstained with hematoxylin (Baso, Zhuhai, China) and dyed blue.

For hematoxylin and eosin (H&E) staining, cells were initially stained with hematoxylin, followed by differentiation and eosin staining. Images were acquired using a Motic upright fluorescent microscope (PA53 BIO FS6) and analyzed in the Case Viewer software (Version 3.3; 3DHISTECH Ltd., Budapest, Hungary).

For quantification, the Lasso tool was used to draw a curve aligned with the nerve fiber layer of the retina and measured as the length of the retina. The GFAP-positive nerve fiber layer length was measured, summed, and normalized to the overall length of the retina. Total Iba1-positive cells were counted in the whole outer nuclear layer and inner nuclear layer areas of analyzed retinal sections and also normalized to the length of the retina.

### 2. Results

Mice bearing the Alpk1⁻ allele from which exon 13 of the murine Alpk1 gene had been deleted (thus abolishing the kinase activity of the encoded protein) were used to prepare ROSAH model mice. The coding sequences (CDS) of wild-type human ALPK1 or human ALPK1 bearing the pThr237Met mutation were inserted immediately following the start codon in exon 2 of murine Alpk1⁻ using CRISPR and homologous recombination techniques, yielding the respective Alpk1^{hALPK1} and Alpk1^{hALPK1T237M} alleles. This enables the generation of homozygous hALPK1 knock-in (hALPK1-KI) mice and mice heterozygous for the hALPK1 and hALPK1^{T237M} alleles (hALPK1^{[T237M]}-KI mice) (FIG. 14). Mice homozygous for Alpk1^{hALPK1} were designated as hALPK1-KI mice, while mice heterozygous for Alpk1^{hALPK1} and Alpk1^{hALPK1T237M}, mimicking the genetic profile of ROSAH patients, were designated as hALPK1^{[T237M]}-KI mice.

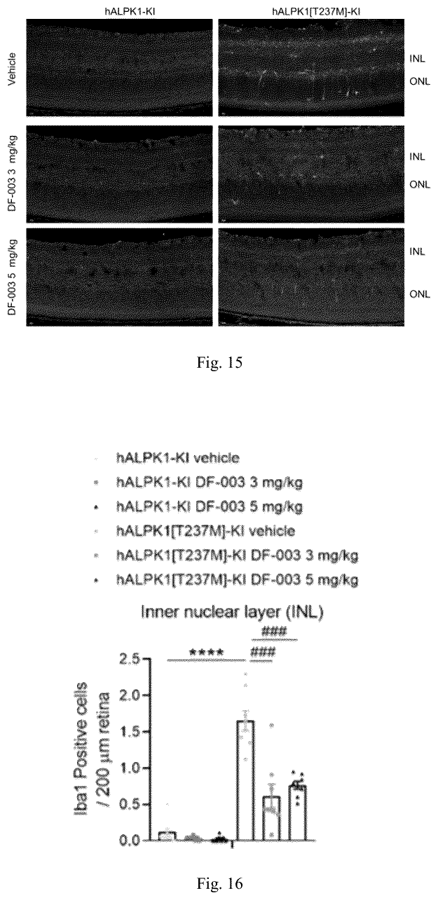

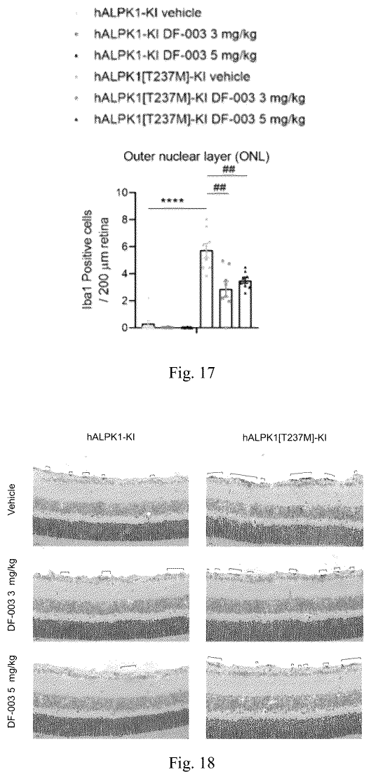

hALPK1^{[T237M]}-KI mice exhibited a significant increase in the numbers of detected microglia (Iba1⁺ cells) in both the outer nuclear layer (ONL) and inner nuclear layer (INL) of the retina as compared to hALPK1-KI control mice, and DF-003 (3 mg/kg) suppressed 68% and 53% of this microglial activation in the INL and ONL, respectively (FIG. 15-17). Data represent means±SEM of each genetic and treatment group. Statistical comparisons between vehicle groups of the two genotypes were made using two-tailed unpaired Student's 1-tests: ***p<0.001; and comparisons between DF-003 dosed groups and vehicle group within the same genotype were made with one-way ANOVAs: ^{##}p<0.01, ^{###}p<0.001.

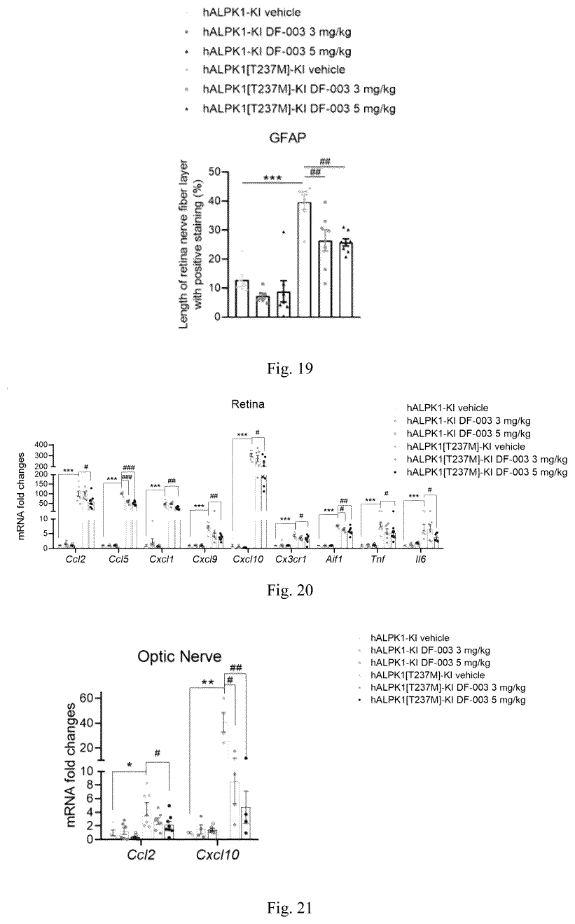

Interestingly, astrocyte activation was similarly detected in retinal cross-sections from these mice by immunohistochemical staining for glial fibrillary acidic protein (GFAP). A significant increase in the frequency of GFAP positivity was detected in the nerve fiber layer of ROSAH model mice as compared to ALPK1-KI controls, and the administration of DF-003 (3 mg/kg) suppressed 49% of the increase in astrocyte activity (FIG. 18-19). Data represent means±SEM for each genetic and treatment group. Statistical comparisons between vehicle groups of the two genotypes were made using two-tailed unpaired Student's t-tests: ***p<0.001; and comparisons between DF-003 dosed groups and the vehicle group within the same genotype were made with one-way ANOVAs: ^{##}p<0.01.

Therefore, DF-003 suppresses retinal microglia infiltration and astrocyte activation in a mouse model of ROSAH syndrome.

### Example 8

### Inhibition of the Upregulation of Pro-Inflammatory Cytokines in the Retina, Optic Nerve, and Brain Cortex of Rosah Model Rodents

### 1. Experiment Method

Female hALPK1-KI and hALPK1^{[T237M]}-KI mice were treated orally with DF-003 once per day for 10 days at the indicated doses. The following day animals were euthanized and the mRNA levels of the indicated genes in the retina, optic nerve, and brain cortex samples from these mice were analyzed by qPCR and normalized to levels of Rpl13.

16- to 17-week-old female hALPK1-KI or hALPK1^{[T237M]}-KI mice were orally administered with 3 mg/kg or 5 mg/kg DF-003, once a day, for 10 days. The body weight of each mouse was measured daily immediately before compound injection. Body weight changes compared to the start of the study were represented as % changes. Data represent mean±SEM for each genotype and treatment group.

### 2. Result

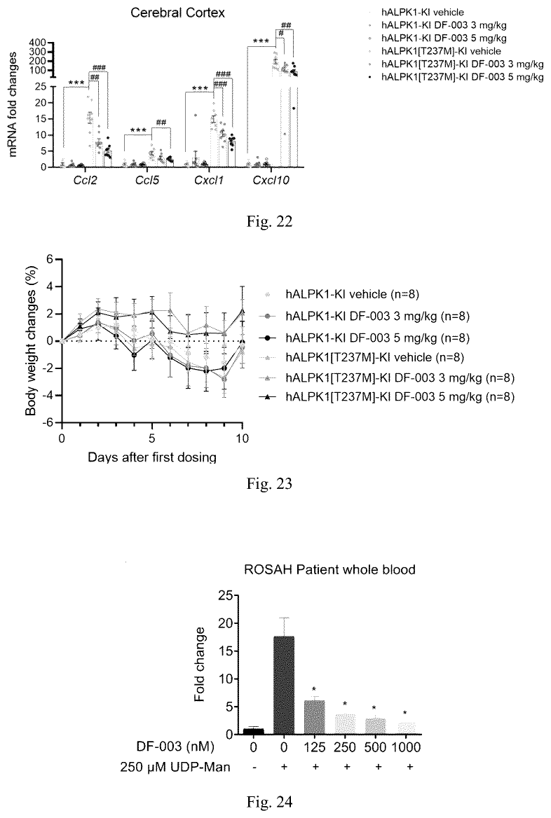

In the retina, Ccl2, Ccl5, Cxcl1, Cxcl9, Cxcl10, Tnf, Il6, as well as the microglia marker genes Cx3cr1 and Aif1, were all upregulated in hAPLK1^{[T237M]}-KI mice (FIG. 20-22), and daily oral administration of DF-003 for 10 days suppressed the upregulation of these genes. Strikingly, Cxcl10, Ccl2, and Ccl5 were upregulated by 305-, 102-, and 102-fold in hALPK1^{[T237M]}-KI mice relative to hALPK1-KI controls, and DF-003 (5 mg/kg) suppressed their induction by 31%, 40%, and 50%, respectively. Notably, the optic nerves of ROSAH model mice exhibited 4.5- and 41-fold increases in the expression of Ccl2 and Cxcl10, and DF-003 (5 mg/kg) suppressed their induction by 66% and 91%, respectively. These changes were not restricted to the retina or optic nerve, as 15-, 4.4-, 15-, and 196-fold increases in Ccl2, Ccl5, Cxcl1, and Cxcl10 expression were also observed in cortical samples from the brains of hALPK1^{[T237M]}-KI mice relative to hALPK1-KI controls, and DF-003 (5 mg/kg) suppressed their corresponding induction by 71%, 56%, 49% and 55%, respectively. These results demonstrate the ability of DF-003 to inhibit ALPK1^{[T237M]}-induced cytokine expression in this mouse model of ROSAH syndrome.

Data represent means±SEM for each genetic and treatment group. Statistical comparisons between vehicle groups of the two genotypes were made using two-tailed unpaired Student's t-tests: *p<0.05, **p<0.01, ***p<0.001, and comparisons between DF-003 dosed groups to the vehicle group within the same genotype were made with one-way ANOVAs: ^{#}p<0.05, ^{##}p<0.01, ^{###}p<0.001.

In general, DF-003 treatments that resulted in the downregulation of cytokine expression were well-tolerated as no significant changes in body weight were observed throughout the course of these experiments (FIG. 23).

### Example 9

### DF-003 Dose-Dependently Inhibits UDP-Mannose-Induced Cytokine Production in ROSAH Patient Whole Blood

To confirm that DF-003 could inhibit ALPK1[T237M]-induced cytokine production in a relevant translational human system, we obtained samples of whole blood from a single ROSAH patient harboring the ALPK1^{T237M} mutation and from the unaffected mother.

### 1. Experiment Method

Whole blood was collected from a ROSAH patient and the unaffected healthy mother using heparin as an anticoagulant. A total of 300 μL of whole blood was added per well of a 48-well culture plate. Various concentrations of DF-003 (125 nM to 1000 nM) or vehicle control (0.1% DMSO) were added to the blood, followed by the addition of 250 μM of acetylated UDP-mannose. Following a 6 h incubation, plasma was separated by centrifugating the blood from each well (10 min, 3,200×g). CXCL8 levels content in the plasma were measured by ELISA using a commercial kit (#555244, BD Biosciences, Franklin Lakes, NJ, USA).

### 2. Results

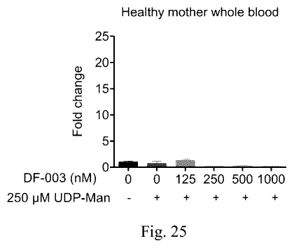

We observed that acetylated UDP-mannose treatment was sufficient to induce the secretion of CXCL8 from cells in ROSAH patient whole blood without any corresponding effect in maternal whole blood (FIG. 24-25). Importantly, DF-003 treatment was sufficient to inhibit this CXCL8 induction in a dose-dependent manner in ROSAH patient whole blood, with an IC₅₀ of 42 nM. Plasma CXCL8 levels were evaluated by ELISA. Fold change vs. 0 nM DF-003 without UDP-Man. *p<0.05, Student's t-test.

### Example 10

### Assessment of Safety and Pharmacokinetics (PK) in Healthy Volunteers (HVs) After Single and Multiple Doses

ROSAH (retinal dystrophy, optic nerve edema, splenomegaly, anhidrosis, and headache) syndrome is a rare genetic disease driven by a gain-of-function ALPK1 T237M mutation with no approved drug treatment. Patients go blind from retinal degeneration, retinal inflammation, and optic atrophy. We developed a first-in-class ALPK1 kinase inhibitor, DF-003, as a precision medicine to target the root cause of ROSAH syndrome. This first-in-human study assessed safety and pharmacokinetics (PK) in healthy volunteers (HVs) after single and multiple doses, including food effect, to support a Phase 2 study in ROSAH patients.

### 1. Experiment Method

This randomized, double-blind, placebo (PBO)-controlled study was conducted in 48 normal HVs meeting inclusion/exclusion criteria. HVs were randomized 6:2 to receive oral DF-003 or matching PBO in 5 single ascending dose (SAD) cohorts (3 to 150 mg) and a 14-day once-daily multiple dose (MD) cohort (50 mg QD) under fasted conditions. Food effect (high-fat meal) was evaluated after a single 20 mg dose in a crossover fashion. Safety and tolerability were assessed by adverse event (AE) monitoring, clinical laboratory/hematology testing, physical exams, vital signs, ECGs, and eye exams. Plasma samples were collected for PK analyses. DF-003 was quantified with a validated LC-MS/MS assay.

### 2. Results

DF-003 was well tolerated in all cohorts; no serious AEs were reported. Single-case AEs were mild (fatigue, headache) and single-case lab abnormalities (creatine kinase, ALT, AST, and LDH) resolved without intervention.

Median time to Cₘₐₓ ranged from 7 to 24 h postdose from 3 to 150 mg in SAD and on Days 1 and 14 in MD. Sampling intervals were extended in SAD cohorts 4 and 5 and in MD to enable determination of terminal t½ (mean: 120-150 h). SAD Cₘₐₓ and AUC₀₋₉₆ₕ increased dose proportionally from 3 to 150 mg; geometric mean (CV %) ranged from 1.0 (31) to 55 (48) ng/ml and 76 (27) to 3520 (36) ng·h/mL, respectively. Food increased Cₘₐₓ and AUC₀₋₉₆ₕ by approximately 30%. Accumulation was 10× to 12× after 14 days of QD dosing.

In this study with HVs, DF-003 was found to be safe without any systemic or ocular complications. PK characterization has provided valuable data to inform selection of a once-daily dose regimen for the continued study of DF-003 to prevent vision loss in patients with ROSAH syndrome.

1. A method for inhibiting ALPK1, comprising administering a pharmaceutically effective amount of a compound of formula (I) or a pharmaceutically acceptable salt thereof,

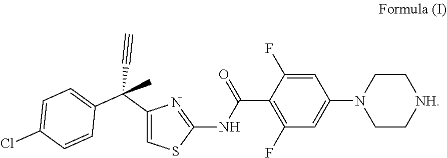 [SMILES: C#C[C@@](C)(c1ccc(Cl)cc1)c1csc(NC(=O)c2c(F)cc(N3CCNCC3)cc2F)n1]

2. The method according to claim 1, wherein the method satisfies one or more of the following conditions:
(1) the method is conducted in vitro, ex vivo, or in vivo;
(2) the ALPK1 is activated by ADPH or D-glycero-D-manno-6-fluoro-heptose-1β-S-ADP;
(3) the ALPK1 is inhibited with an IC₅₀ of 0.75-4 nM, such as 1.5 nM;
(4) the ALPK1 is inhibited with 400-1600 fold selectivity over TAOK2/TAO1; such as 860-fold;
(5) the ALPK1 is inhibited with 1000-4000 fold selectivity over CAMK2g; such as 2067-fold;
(6) the ALPK1 is inhibited with 1500-6000 fold selectivity over CAMK1a; such as 3067-fold.

3. A method for reducing the levels of inflammatory cytokine and chemokine medicated by ALPK1, comprising administering a pharmaceutically effective amount of the compound of formula (I) according to claim 1 or a pharmaceutically acceptable salt thereof.

4. The method according to claim 3, wherein the method satisfies one or more of the following conditions:
(1) the method is conducted in vitro, ex vivo, or in vivo;
(2) the inflammatory cytokine and chemokine is selected from TNF and CXCL8; preferably, the inflammatory cytokine and chemokine is activated by ADPH or D-glycero-D-manno-6-fluoro-heptose-1β-S-ADP;
(3) when the method is conducted in vitro, the method is conducted in cells, such as THP-1 macrophages, HEK-293 cells stably overexpressing ALPK1 or NF-κB reporter cells overexpressing ALPK1; preferably, the cells are triggered by ADPH or D-glycero-D-manno-6-fluoro-heptose-1β-S-ADP.

5. The method according to claim 3, wherein the method satisfies one or more of the following conditions:
(1) when the inflammatory cytokine and chemokine is TNF in THP-1 macrophages triggered by D-glycero-D-manno-6-fluoro-heptose-1β-S-ADP, the TNF upregulation is inhibited with an IC₅₀ of 4-15 nM, such as 8 nM, in the form of mRNA fold change;
(2) when the inflammatory cytokine and chemokine is CXCL8 in THP-1 macrophages triggered by D-glycero-D-manno-6-fluoro-heptose-1β-S-ADP, the CXCL8 upregulation is inhibited with an IC₅₀ of 3-12 nM, such as 6.5 nM, in the form of mRNA fold change.

6. A method for inhibiting ALPK1^{[T237M]}, comprising administering a pharmaceutically effective amount of a compound of formula (I) or a pharmaceutically acceptable salt thereof,

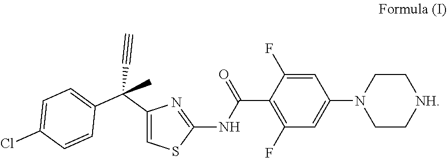 [SMILES: C#C[C@@](C)(c1ccc(Cl)cc1)c1csc(NC(=O)c2c(F)cc(N3CCNCC3)cc2F)n1]

7. The method according to claim 6, wherein the method satisfies one or more of the following conditions:
(1) the method is conducted in vitro, ex vivo, or in vivo;
(2) the ALPK1^{[T237M]} is activated by ADPH or UDP-mannose;
(3) when the ALPK1^{[T237M]} is activated by UDP-mannose, UDP-mannose-stimulated ALPK1^{[T237M]} is inhibited in a dose-dependent manner with an IC₅₀ of 8-30 nM, such as 16 nM.

8. A method for reducing the levels of inflammatory cytokine and chemokine medicated by ALPK1^{[T237M]}, comprising administering a pharmaceutically effective amount of the compound of formula (I) according to claim 6 or a pharmaceutically acceptable salt thereof,

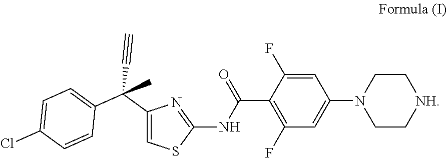 [SMILES: C#C[C@@](C)(c1ccc(Cl)cc1)c1csc(NC(=O)c2c(F)cc(N3CCNCC3)cc2F)n1]

9. The method according to claim 8, wherein the method satisfies one or more of the following conditions:
(1) the method is conducted in vitro, ex vivo, or in vivo;
(2) when the method is conducted in vitro, the method is conducted in cells, such as HEK-293 cells stably overexpressing ALPK1^{[T237M]} or NF-κB reporter cells overexpressing ALPK1^{[T237M]};
(3) the inflammatory cytokine and chemokine is selected from TNF, CXCL10, and CXCL8; or, the inflammatory cytokine and chemokine is selected from Ccl2, Ccl5, Cxcl1, Cxcl9, Cxcl10, Tnf, Il6, Cx3cr1 and Aif1;
(4) the inflammatory cytokine and chemokine is upregulated by UDP-mannose.

10. The method according to claim 8, wherein the method satisfies one or more of the following conditions:
(1) when the method is conducted in NF-κB reporter cells overexpressing ALPK1^{[T237M]}, NF-κB reporter activity is inhibited in a dose-dependent manner;
(2) when the method is conducted in HEK-293 cells stably overexpressing ALPK1^{[T237M]}, TNF, CXCL10, and CXCL8 expressions are inhibited in a dose-dependent manner.

11. The method according to claim 10, wherein the method satisfies one or more of the following conditions:
(1) when the method is conducted in HEK-293 cells stably overexpressing ALPK1^{[T237M]}, CXCL10 is inhibited with an IC₅₀ of 70-260 nM, such as 134 nM, in the form of mRNA fold change;
(2) when the method is conducted in HEK-293 cells stably overexpressing ALPK1^{[T237M]}, TNF is inhibited with an IC₅₀ of 45-180 nM, such as 92 nM, in the form of mRNA fold change;
(3) when the method is conducted in HEK-293 cells stably overexpressing ALPK1^{[T237M]}, CXCL8 is inhibited with an IC₅₀ of 70-260 nM, such as 136 nM, in the form of mRNA fold change;
(4) when the method is conducted in HEK-293 cells stably overexpressing ALPK1^{[T237M]}, the enhanced secretion of CXCL8 from HEK-293 cells overexpressing ALPK1^{T237M} is inhibited with an IC₅₀ of 60-250 nM, such as 125 nM.

12. The method according to claim 8, wherein the method satisfies one or more of the following conditions:
(1) when the method is conducted in vivo, a subject is a mouse model of ROSAH syndrome; such as a mouse heterozygous for the hALPK1 and hALPK1^{T237M} alleles;
(2) when the method is conducted in vivo, a subject is a ROSAH patient;
(3) when the method is conducted in vivo, the compound of formula (I) is administered orally;
(4) when the method is conducted in vivo, the compound of formula (I) is administered at 2-10 mg/kg, once per day; such as 2, 3, 5 or 10 mg/kg, once per day;
(5) when the method is conducted in vivo, the compound of formula (I) is administered for 5-10 days.

13. The method according to claim 8, wherein the method satisfies one or more of the following conditions:
(1) when the method is conducted in vivo, Cxcl10, Ccl2, and Ccl5 expressions in retina in ROSAH model mice are inhibited by 30-50%, such as 31%, 40%, and 50%, respectively, in the form of mRNA fold change, with the compound of formula (I) once per day for 10 days at the dose of 5 mg/kg;
(2) when the method is conducted in vivo, Ccl2 and Cxcl10 expressions in optical nerves in ROSAH model mice are inhibited by 60-95%, such as 66% and 91%, respectively, in the form of mRNA fold change, with the compound of formula (I) once per day for 10 days at the dose of 5 mg/kg;
(3) when the method is conducted in vivo, Ccl2, Ccl5, Cxcl1, and Cxcl10 expressions in cortical samples from the brains in ROSAH model mice are inhibited by 45-75%, such as 71%, 56%, 49% and 55%, respectively, in the form of mRNA fold change, with the compound of formula (I) once per day for 10 days at the dose of 5 mg/kg;
(4) when the method is conducted in vivo, CXCL8 induction is inhibited in a dose-dependent manner in ROSAH patient whole blood with an IC₅₀ of 20-80 nM, such as 42 nM; preferably, CXCL8 is induced by acetylated UDP-mannose.

14. A method for inhibiting microglia or astrocyte activation in a subject in need thereof, comprising administering a pharmaceutically effective amount of the compound of formula (I) according to claim 6 or a pharmaceutically acceptable salt thereof.

15. The method according to claim 14, wherein the method satisfies one or more of the following conditions:
(1) the compound of formula (I) is administered orally;
(2) the compound of formula (I) is administered at 2-10 mg/kg, once per day; such as 2, 3, 5 or 10 mg/kg, once per day;
(3) the compound of formula (I) is administered for 5-10 days;
(4) the subject is a mouse model of ROSAH syndrome; such as a mouse heterozygous for the hALPK1 and hALPK1^{T237M} alleles.

16. The method according to claim 14, wherein the method satisfies one or more of the following conditions:
(1) the microglia is the microglia in both the outer nuclear layer and inner nuclear layer of the retina;
(2) the astrocyte is the astrocyte in retina nerve fiber layer;
(3) microglial activation in the INL and ONL are inhibited by 50-70% in ROSAH model mice, such as 68% and 53%, respectively, with the compound of formula (I) once per day for 10 days at the dose of 3 mg/kg;
(4) astrocyte activity is inhibited by 35%-60%, such as 49% in ROSAH model mice, with the compound of formula (I) once per day for 10 days at the dose of 3 mg/kg.

17. A method for treating ROSAH syndrome in a subject in need thereof, comprising administering a pharmaceutically effective amount of a compound of formula (I) or a pharmaceutically acceptable salt thereof,

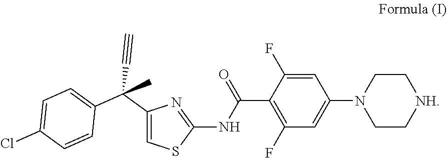 [SMILES: C#C[C@@](C)(c1ccc(Cl)cc1)c1csc(NC(=O)c2c(F)cc(N3CCNCC3)cc2F)n1]

18. The method according to claim 17, wherein the method satisfies one or more of the following conditions:
(1) the compound of formula (I) is administered orally;
(2) the compound of formula (I) is administered at 2-10 mg/kg, once per day; such as 2, 3, 5 or 10 mg/kg, once per day;
(3) the compound of formula (I) is administered for 5-10 days;
(4) the compound of formula (I) crosses the blood-retina barrier, or blood-brain barrier;
(5) the method is safe without any systemic or ocular complications.

19. The method according to claim 17, wherein the method satisfies one or more of the following conditions:
(1) when the compound of formula (I) is administered at 3 or 5 mg/kg once per day for 10 days, maximum plasma concentrations of the compound of formula (I) ranges from 160 and 310 ng/ml after 10^{th} daily oral dosing;
(2) when the compound of formula (I) is administered at 3 or 5 mg/kg once per day for 10 days, an average time to Cₘₐₓ ranges from 2-2.67 h after 10^{th} daily oral dosing;
(3) when the compound of formula (I) is administered at 3 or 5 mg/kg once per day for 10 days, measured half-life values for the compound of formula (I) in the 3 and 5 mg/kg groups following the 10^{th} dose is 7.69 and 8.81 h, respectively.


SEQUENCE LISTING (16 sequences)

SEQ ID NO: 1 - protein, 23 aa, synthetic construct
MDYKDHDGDYKDHDIDYKDDDDK

SEQ ID NO: 2 - DNA, 44 bp, synthetic construct
  source 1..44: mol_type = other DNA
ctcttctgcctgctgcactttgatgggctacaggcttgtcactc

SEQ ID NO: 3 - DNA, 45 bp, synthetic construct
  source 1..45: mol_type = other DNA
ggtgagaagagatgtctgaatccgtccatccttggaagcactgca

SEQ ID NO: 4 - DNA, 40 bp, synthetic construct
  source 1..40: mol_type = other DNA
aaggtgcagttttgccaaggcccagttttccttggggtcc

SEQ ID NO: 5 - DNA, 40 bp, synthetic construct
  source 1..40: mol_type = other DNA
aattccatggcaccgtcaagtggactccacgacgtactca

SEQ ID NO: 6 - DNA, 42 bp, synthetic construct
  source 1..42: mol_type = other DNA
ccaatgagtaggctggagagcgacccattccttcttggggtc

SEQ ID NO: 7 - DNA, 40 bp, synthetic construct
  source 1..40: mol_type = other DNA
ctcaccatatggctcggacacgactgcaagattggagcac

SEQ ID NO: 8 - DNA, 40 bp, synthetic construct
  source 1..40: mol_type = other DNA
actcaagaatggtcgcgagggtgccatcagagcagtctgt

SEQ ID NO: 9 - DNA, 47 bp, synthetic construct
  source 1..47: mol_type = other DNA
cctagtgataaggaatgcacgatgctaggcaggtttgatctccgttc

SEQ ID NO: 10 - DNA, 41 bp, synthetic construct
  source 1..41: mol_type = other DNA
ccacgtgttgagatcattgccgaggctctctgctgtccatc

SEQ ID NO: 11 - DNA, 40 bp, synthetic construct
  source 1..40: mol_type = other DNA
gtgagtgactggcacttcctgaccgaacgtgaagacgagg

SEQ ID NO: 12 - DNA, 44 bp, synthetic construct
  source 1..44: mol_type = other DNA
tctgccgtccaaacttgaagccctcttcagctctaggtgggtct

SEQ ID NO: 13 - DNA, 42 bp, synthetic construct
  source 1..42: mol_type = other DNA
ccctcacactcagatcatcttctgctacgacgtgggctacag

SEQ ID NO: 14 - DNA, 44 bp, synthetic construct
  source 1..44: mol_type = other DNA
ctgctctcaaggttgttcggctccttccgtttctcctccagagt

SEQ ID NO: 15 - DNA, 80 bp, synthetic construct
  source 1..80: mol_type = other DNA
gggaatttccgggaatttccgggaatttccgggaatttccgggaatttccgggaatttccgggaatttccgggaatttcc

SEQ ID NO: 16 - RNA, 23 nt, synthetic construct
  source 1..23: mol_type = other RNA
agtgaggaccagcggtgcagagg